(19) United States
(12) Patent Application Publication
(10) Pub. No.: US 2006/0223108 A1
(43) Pub. Date: Oct. 5, 2006

(54) Methods and kits for diagnosing and monitoring hematopoietic cancers

(75) Inventors: Iris Pecker, Rishon LeZion (IL); Israel Vlodavsky, Mevaseret Zion (IL); Ben Zion Katz, Rehovot (IL); Amiram Eldor, Tel Aviv (IL); Menachem Bitan, Jerusalem (IL); Aaron Polliack, Jerusalem (IL); Arnon Nagler, Jerusalem (IL); Zofia Eldor, Tel Aviv (IL)

Correspondence Address: Martin D. Moynihan;PRTSI, Inc., P.O. Box 16446, Arlington, VA, 22215, US

(73) Assignees: Hadasit Medical Research Services and Development Ltd.; Insight Strategy & Marketing Ltd.

(21) Appl. No.: 11/449,775

(22) Filed: Jun. 9, 2006

Related U.S. Application Data
(63) Continuation of application No. 10/227,544, filed on Aug. 26, 2002.
(60) Provisional application No. 60/314,302, filed on Aug. 24, 2001.

(51) Int. Cl. C12Q 1/68; G01N 33/574; C12Q 1/34

(52) U.S. Cl. 435/6; 435/7.23; 435/18

(57) ABSTRACT

Methods and kits for distinguishing between heparanase expressing and heparanase non-expressing hematopoietic cells, particularly useful in distinguishing between different types of differentiated and/or undifferentiated lymphomas and leukemias. The methods and kits are designed to detect heparanase expression, or absence thereof, at the gene, protein and/or protein activity levels.

This Application claims the benefit of priority from U.S. Provisional Patent Application No. 60/314,302, filed Aug. 24, 2001.

### FIELD AND BACKGROUND OF THE INVENTION

The present invention relates to a method and kits for diagnosing and monitoring hematopoietic cancers. More particularly, the present invention relates to methods and kits for distinguishing between different types of differentiated and/or undifferentiated lymphomas and leukemias, and diagnostic applications thereof. Most particularly, the present invention relates to methods and kits for distinguishing between acute myeloid leukemia cells and chronic myeloid leukemia cells, B cell leukemia and B cell lymphoma based on the presence or absence of heparanase expression in blood or bone marrow cells obtained from a patient, whereby heparanase expression serves as a target for diagnostics.

Heparan sulfate proteoglycans (HSPG) are macromolecules composed of a core protein covalently O-linked to repeating hexuronic and D-glucosamine disaccharide units (1-3). HSPG are located at the sub-endothelial basement membrane, where they cross-link a variety of extracellular matrix (ECM) molecules (e.g., collagen, laminin, fibronectin), thereby contributing to the formation and preservation of blood-vessels walls (4). Circulating blood cells extravasate to an inflammatory focus by degrading various components of the sub-endothelial basement membrane, in response to stimuli (5). Circulating metastatic tumor cells exploit this pathway, and similarly degrade sub-endothelial basement membrane, to form additional tumor foci (6). Metastatic spread of blood-borne tumor cells involves degradation of blood vessel wall constituents including basement membrane molecules such as collagen type IV, laminin and HSPG (9-12, 20, 21). Hence, malignant cells constitutively express a variety of ECM-degrading enzymes, including members of the matrix metalloproteinase (MMP) family (e.g., MMP-2 and MMP-9) (7, 20, 22-24). MMP-2 and MMP-9 are produced by lymphoid and myeloid neoplasias as well as by normal hematopoietic cells, including granulocytes, monocytes and lymphocytes (8).

HSPG degradation disintegrates the sub-endothelial basement membrane, consequently facilitating trans-endothelial migration (extravasation) of blood-borne cells (9, 10). Heparanase is an endo-β-D-glucuronidase, degrading the heparan sulfate (HS) side chains of HSPG. Heparanase activity has been illustrated in several types of normal hematopoietic cells, including neutrophils, megakaryocytes and activated lymphocytes, and may mediate their extravasation during immune responses (10). Heparanase expression has also been shown to correlate with the metastatic potential of a number of tumor cells, including lymphoma, fibrosarcoma, breast carcinoma and melanoma cells (9, 11-15). However, the heparanase expression pattern during leukemic hematopoiesis has not been defined.

Recently, the gene encoding the human heparanase protein was cloned and its deduced amino acid sequence includes a potential signal peptide, indicating that heparanase can be secreted outside the plasma membrane (14-17 and U.S. Pat. No. 5,968,822). The heparanase genomic locus was found to span approximately 40 kb and localized on the human chromosome 4q21.3 (14, 15, and U.S. Pat. No. 5,968,822). Transfection of heparanase cDNA into non-metastatic lymphoma cells resulted in an increase in their metastatic potential, demonstrating a direct link between heparanase production and the malignancy of hematopoietic cells (14).

Previous studies evaluated the expression of MMPs in malignant hematopoietic cells (8). MMP-9 expression was detected in bone marrow samples obtained from normal individuals and patients with several hematological disorders, including AML, CML and MDS (8). MMP-2 expression was detected predominantly in AML and MDS, but not in normal or CML bone marrow samples (8). Thus a marker protein facilitating the distinction between the various types of malignant hematopoietic cells from each other, and from normal cells, is clearly lacking, and of critical importance.

There is thus a widely recognized need for, and it would be highly advantageous to have, a method of distinguishing between malignant hematopoietic cells and normal cells, yielding unambiguous results. A method utilizing a single marker would provide an enormous breakthrough in the diagnosis of hematopoietic cell malignancies.

Though heparanase expression in aberrant hematopoietic cells is an attractive candidate for distinguishing between malignant hematopoietic cells and normal cells, heparanase has not, to date, been investigated in this context.

### SUMMARY OF THE INVENTION

The present invention discloses a method and kit for distinguishing between heparanase expressing and heparanase non-expressing hematopoietic cells. Furthermore, the present invention discloses the use of heparanase expression for distinguishing between differentiated and/or undifferentiated lymphomas and leukemias and their relapse. The present invention further discloses a method and kit for ascertaining the suitability of an anti-neoplastic drug candidate for efficacy in treating leukemias, based on altered heparanase expression. Heparanase expression may be evaluated at the gene, protein or activity level, and methods and kits detecting heparanase expression can therefore be utilized for diagnostic applications. Because heparanase RNA, protein and activity were not detected in most other leukemias, or hematoncological disorders, heparanase can be utilized as a single marker for acute myelogenous leukemias, independent of more common membrane markers, or alternatively in combinations thereof.

It is therefore one object of the present invention to provide a method of distinguishing between heparanase expressing and heparanase non-expressing hematopoietic cells.

It is yet another object of the present invention to provide a method of distinguishing between heparanase expressing and heparanase non-expressing neoplastic and normal hematopoietic cells.

It is still yet another object of the present invention to provide a method of ascertaining the suitability of an anti-neoplastic drug candidate for efficacy in treating acute myelogenous leukemias.

It is still another object of the present invention to provide kits for distinguishing between heparanase expressing and heparanase non-expressing normal and neoplastic hematopoietic cells.

Thus, according to one aspect of the present invention there is provided a method of distinguishing between heparanase expressing and heparanase non-expressing hematopoietic cells in a subject in need thereof, the method comprising monitoring heparanase expression in hematopoietic cells obtained from the subject and, based on the presence or absence of heparanase expression, distinguishing between heparanase expressing and heparanase non-expressing hematopoietic cells.

According to another aspect of the present invention there is provided an improvement in a process of diagnosing acute myelogenous leukemia or its relapse, versus non-Hodgkin's lymphoma, acute lymphocytic leukemia, chronic lymphocytic leukemia or chronic myeloid leukemia, the improvement comprising determining whether hematopoietic cells suspected as neoplastic and in particular, leukemic cells express heparanase, wherein heparanase expression is indicative of acute myeloid leukemia, and wherein non-expression of heparanse in neoplastic hematopoietic cells is indicative of non-Hodgkin's lymphoma acute lymphocytic leukemia, chronic lymphocytic leukemia or chronic myeloid leukemia.

According to still another aspect of the present invention, there is provided a method for ascertaining the suitability of an anti-neoplastic drug candidate for efficacy in treating acute myeloid leukemia, wherein said method comprises combining said candidate drug with an acute myeloid leukemia cell, said cell being isolated or in a biological sample and detecting in said cell or biological sample a change in heparanase expression.

According to further features in preferred embodiments of the invention described below, the hematopoietic cells assessed for heparanase expression may be obtained from lymph nodes, bone marrow and/or peripheral blood.

According to further features in preferred embodiments of the invention described below, the hematopoietic cells obtained from the subject may be further enriched for leukocytes prior to monitoring heparanase expression.

According to still further features in the described preferred embodiments, heparanase expressing hematopoietic cells may comprise activated T or B lymphocytes, platelets, granulocytes, macrophages, monocytes, mast cells, megakaryocytes, promegakaryocytes, megakaryoblasts, promyelocytes, metamyelocytes, myelocytes, myeloblasts, monoblasts, and dendritic cells.

According to still further features in the described preferred embodiments, heparanase non-expressing hematopoietic cells may comprise erythrocytes, normoblasts, erythroblasts hematopoietic stem cells and naive T and B lymphocytes.

According to still further features in the described preferred embodiments, hematopoietic cells may additionally express the surface markers CD2, CD3, CD4, CD7, CD8, CD10, CD11b, CD13 CD14, CD15, CD16, CD18, CD19, CD20, CD22, CD23, CD25, CD28, CD33, CD34, CD38, CD41, CD42a, CD42b, CD45, CD61, CD71, CD103, CD117, HLA-DR, MPO, FMC7 and/or TdT.

According to still further features in the described preferred embodiments, the method may be used to distinguish between heparanase expressing myeloid cells (AML) and heparanase non-expressing myeloid leukemia cells (CML) and/or between heparanase expressing myeloid cells and heparanase non-expressing B lymphoma cells and/or between heparanase expressing myeloid cells and heparanase non-expressing B leukemia cells.

According to still further features in the described preferred embodiments, the method may be used to distinguish between heparanase expressing acute myeloid leukemia cells and heparanase non-expressing chronic and/or acute lymphocytic leukemia cells or between heparanase expressing acute myeloid leukemia cells and heparanase non expressing chronic myeloid leukemia cells and between heparanase expressing acute myeloid leukemia cells and heparanase non expressing non-Hodgkin's lymphoma cells.

According to still further features in the described preferred embodiments, monitoring heparanase expression in hematopoietic cells obtained from the subject is by monitoring heparanase RNA levels.

According to still further features in the described preferred embodiments, monitoring heparanase RNA levels may be effected by an assay selected from the group consisting of RT-PCR, chip hybridization, RNase protection, in-situ hybridization, primer extension, Northern blot and dot blot analysis.

According to still further features in the described preferred embodiments, monitoring heparanase expression in hematopoietic cells obtained from the subject is by monitoring heparanase protein level.

According to still further features in the described preferred embodiments, monitoring heparanase protein levels may be effected by an assay selected from the group consisting of immunohistochemistry, ELISA, RIA, Western blot analysis, FACS analysis, an immunofluorescence assay, and a light emission immunoassay.

According to still further features in the described preferred embodiments, monitoring heparanase expression in hematopoietic cells obtained from the subject is by monitoring heparanase activity levels.

According to still further features in the described preferred embodiments, monitoring heparanase activity levels may be effected by using a heparanase substrate.

According to still further features in the described preferred embodiments, heparanase substrates may be selected from the group consisting of heparan sulfate proteoglycans, soluble or immobilized heparan sulfate, or heparin, and may include a detectable moiety selected from the group consisting of a chromogenic moiety, a fluorogenic moiety, a radioactive moiety and a light-emitting moiety.

According to another aspect of the present invention there is provided a kit for distinguishing between heparanase expressing hematopoietic and heparanase non-expressing hematopoietic cells, comprising at least one container including at least one reagent for determining a level of heparanase expression or activity in a biological sample, and packaging material identifying said at least one reagent for use in distinguishing between heparanase expressing hematopoietic cells and heparanase non-expressing hematopoietic cells.

According to another aspect of the present invention there is provided a kit for distinguishing between heparanase expressing acute myeloid leukemic cells and/or other neoplastic or normal hematopoietic cells and heparanase non-expressing non-Hodgkin's lymphoma cells and/or chronic lymphocytic leukemia cells and/or chronic myeloid leukemic cells and/or other neoplastic or normal hematopoietic cells, comprising at least one container including at least one reagent for determining a level of heparanase expression or activity in a biological sample, and packaging material identifying said at least one reagent for use in distinguishing between the above listed heparanase expressing cells and the above listed heparanase non-expressing neoplastic or normal hematopoietic cells.

According to still further features in the described preferred embodiments the reagent comprises a substance selected for specifically interacting with heparanase mRNA, for monitoring heparanase RNA levels within said cells.

According to still further features in the described preferred embodiments heparanase RNA levels are monitored via an assay selected from the group consisting of RT-PCR, chip hybridization, RNase protection, in-situ hybridization, primer extension, Northern blot and dot blot analysis.

According to still further features in the described preferred embodiments the reagent may comprise oligonucleotide primers for the identification and/or amplification of heparanase mRNA.

According to still further features in the described preferred embodiments the reagent comprises a substance selected for specifically interacting with heparanase, for monitoring heparanase protein levels or activity within said cells.

According to still further features in the described preferred embodiments heparanase protein levels are monitored via an assay selected from the group consisting of immunohistochemistry, ELISA, RIA, Western blot analysis, FACS analysis, an immunofluorescence assay, and a light emission immunoassay, said at least one reagent comprises a heparanase specific antibody.

According to still further features in the described preferred embodiments the reagent may comprise a heparanase specific antibody, and may be coupled to an enzyme.

According to still further features in the described preferred embodiments the reagent may comprise a heparanase substrate.

According to still further features in the described preferred embodiments the heparanase substrate may be selected from the group consisting of soluble or immobilized heparan sulfate proteoglycans, heparan sulfate, or heparin, and may additionally comprise a detectable moiety selected from the group consisting of a chromogenic moiety, a fluorogenic moiety, a radioactive moiety and a light-emitting moiety.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a photograph of an agarose gel electrophoresis of products of an RT-PCR assay indicating heparanase RNA expression in peripheral white blood cells of normal donors (n=6). Heparanase-specific primers (yielding a 585 bp PCR product) (lanes 1-6, top) and GAPDH primers (yielding a 598 bp PCT product) (lanes 1-6, bottom) were utilized to probe for expression of the respective mRNAs. A pcDNA3 plasmid containing the full-length heparanase cDNA served as the positive control (lane 7, top), human melanoma cDNA functioned as a control template (lane 7, bottom), and a sample containing reagents alone functioned as the negative control (lane 8). Lane 9: DNA molecular weight.

FIG. 2A is a photograph revealing heparanase expression by immunocytochemistry in peripheral blood leukocytes. Polymorphonuclear cells isolated from normal blood samples revealed strong cytoplasmic (green fluorescent) staining of the polymorphonuclear (PMN) cells, as compared to no detectable heparanase expression in mononuclear (MN) cells and erythrocytes.

FIG. 2B is a chromatogram showing release of ³⁵S-labeled ECM degradation fragments, analyzed by gel filtration, indicating functional heparanase activity. Isolated peripheral blood monocytes were incubated with ECM in the presence (open squares) or absence (closed squares) of 2.5 μg/ml heparin, indicating enzyme specificity.

FIG. 3A is a photograph of an agarose gel electrophoresis of products of an RT-PCR assay indicating heparanase gene expression in leukemic leukocytes. White blood cells isolated from patients with various leukemias were analyzed for heparanase expression. Heparanase transcripts were expressed in leukemic cells from patients with AML exclusively; no expression was detected in samples from patients with CLL, ALL, NHL or CML. The positive control containing a plasmid encoded heparanase gene (hpa pcDNA) yielded a positive signal as well.

FIG. 3B is a photograph revealing heparanase expression as detected by immunohistochemistry. Cell-specific heparanase protein expression was evident as red-brown precipitate staining (indicated by black arrows) of neutrophils (No. 1) and platelets (No. 2), but not lymphocytes (No. 2) (indicated by red arrow) in normal donor samples. Cytoplasmic staining was also evident in AML blasts (No. 5) but not in CML (No. 4) or CLL (No. 3) leukemic cells (indicated by black arrows), while positively stained neutrophils are indicated by red arrow).

FIG. 4 is a chromatogram showing release of ³⁵S-labeled ECM degradation fragments, analyzed by gel filtration, indicating functional heparanase activity in leukemic cells from AML-M4 (open symbols) but not CLL (closed symbols) patients. 2.5×10⁶ AML cells/ml were incubated with ³⁵S-labeled ECM in the absence (open squares) or presence (open circles) of 2.5 μg/ml heparin and analyzed by gel filtration, indicating enzymatic activity. The inset is a photograph of an agarose gel electrophoresis of total RNA assessed by RT-PCR probed with primers specific for heparanase (top) and GAPDH (bottom). AML-M4 (lane 1) and positive control pcDNA3 (containing the heparanase cDNA (lane 3, top)) revealed the presence of the appropriately-sized transcript (molecular weight markers in lane 5), however, the CLL (lane 2), and negative control (lane 4) samples did not.

FIG. 5A is a plot of data analyzed by flow cytometric analysis of cell-surface heparanase expression on the cell surface of the total population of isolated normal circulating leukocytes. Cells probed with FITC-conjugated anti-heparanase mAb revealed modest staining as compared to an isotype-matched control.

FIG. 5B is a plot of data analyzed by flow cytometric analysis of cell-surface heparanase expression of cell populations in (A) sorted by size and granularity (indicated by the regions Gr, Mo and Ly corresponding to granulocytes, monocytes and lymphocytes, respectively). Granulocyte populations revealed very low levels of surface staining for heparanase protein, monocytes revealed appreciably less staining, and lymphocyte staining was not detected.

FIG. 6A is a plot of data analyzed by flow cytometric analysis of M4 AML peripheral leukocyte probed with isotype-matched control monoclonal antibodies, determining background fluorescence.

FIG. 6B is a plot of data analyzed by flow cytometric analysis of M4 AML bone marrow cell populations probed with isotype-matched control monoclonal antibodies, determining background fluorescence.

FIG. 6C is a plot of data analyzed by flow cytometric analysis revealing significant cell-surface heparanase and CD 14 expression in M4 AML peripheral leukocytes, probed with fluorescent-labeled anti-heparanase mAb 130 (indirect immunofluorescence) and fluorescent-labeled anti CD 14 mAb (direct immunofluorescence).

FIG. 6D is a plot of data analyzed by flow cytometric analysis of bone marrow cell populations, taken from patients with M4 AML, prior to (left panel) and following (right panel) clinical remission, probed with fluorescent-labeled anti-heparanase mAb 130 (indirect immunofluorescence) and fluorescent-labeled anti CD14 mAb (direct immunofluorescence). Bone marrow samples stained positively for CD14 cell surface expression, while heparanase expression declined as a function of clinical remission.

FIG. 7 is a schematic diagram depicting the proposed lineage-specific expression of heparanase in cells of hematopoietic origin. Heparanase gene and protein are not expressed in early hematopoietic CD34+ progenitors, as evidenced by the lack of heparanase expression in CML samples as well as in CD34+ isolated cord blood cells (Table 1, below). While cellular activation events may turn-on heparanase expression in non-expressing cells (e.g., lymphocytes), or induce the release of the enzyme from heparanase expressing cells (e.g., neutrophils), the highest levels of expression were more commonly found in leukemic cells at an arrested stage of maturation along the granulocytic differentiation pathway.

### DESCRIPTION OF THE PREFERRED EMBODIMENTS

The present invention is of methods and kits for distinguishing between heparanase expressing and heparanase non-expressing normal and/or neoplastic hematopoietic cells. Specifically, the present invention utilizes heparanase expression in order to distinguish between normal hematopoietic cells and differentiated and/or undifferentiated lymphomas and leukemias and their relapse. Heparanase expression is also used as a means of ascertaining the suitability of anti-neoplastic drug candidates for efficacy in treating leukemias, and in other diagnostic applications. As is exemplified herein, because heparanase RNA, protein and activity were not detected in most other leukemias, or hematoncological disorders, heparanase can be utilized as a single marker for acute myeloid leukemias and independent of more common membrane markers, or alternatively in combinations thereof.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Heparanase is an endo-β-D-glucuronidase, which degrades extracellular matrix (ECM) components consisting of heparan sulfate proteoglycans (HSPG). Heparanase activity has been illustrated in several types of normal hematopoietic cells, including neutrophils, megakaryocytes and activated lymphocytes, mediating their extravasation during immune responses (10). Heparanase expression has also been shown to correlate with the metastatic potential of a number of tumor cells, including lymphoma, fibrosarcoma, breast carcinoma and melanoma cells (9, 11-15). However, the heparanase expression pattern during leukemic hematopoiesis has not been defined, nor has a systematic approach toward differentiating leukemic, lymphoma and normal hematopoietic cell populations based on heparanase expression has been achieved.

Metastatic spread of blood-born tumor cells involves degradation of blood vessel wall constituents including basement membrane molecules such as collagen type IV, laminin and HSPG (8-11, 19, 20). Hence, malignant cells constitutively express a variety of ECM-degrading enzymes, including matrix metalloproteinases (MMPs) and heparanase (7-12, 19, 21-23).

Previous studies evaluated the expression of MMPs in malignant hematopoietic cells (7). MMP-9 expression was detected in bone marrow samples obtained from normal individuals and patients with several hematological disorders, including AML, CML and MDS (7). In contrast, MMP-2 expression was detected predominantly in AML and MDS, but not in normal or CML bone marrow samples (7). Due to the non-availability of appropriate anti-heparanase monoclonal antibodies and molecular probes by most researchers, heparanase expression in aberrant hematopoietic cells has not been sufficiently investigated. Nor has it been possible to differentiate between various hematopoietic tumors, using a single marker protein, a measure that would dramatically impact diagnostic procedures for diagnosing hematopoietic neoplasms.

While reducing the present invention to practice it was discovered that hematopoietic cells, both normal and neoplastic, could be distinguished, based on their expression of heparanase. Neutrophils have been suggested as the major cell type expressing heparanase in circulation (28). The present invention, however, provides a definitive means to assess myeloid-lineage versus, lymphocytic and erythrocytic expression of heparanase.

Furthermore, while reducing the present invention to practice, it was uniquely discovered that acute myelogenous leukemias express high levels of heparanase, whereas lymphomas lymphocytic leukemias and chronic myelogenous leukemia do not. While normal, hematopoietic cells of myeloid lineage, in particular the granulocytes express heparanase; erythrocytes and naive lymphocytes do not.

Therefore, according to one aspect of the present invention there is provided a method of distinguishing between heparanase expressing hematopoietic cells and heparanase non-expressing hematopoietic cells in a subject in need thereof, the method comprising monitoring heparanase expression in hematopoietic cells obtained from the subject and, based on a presence or an absence of said heparanase expression, distinguishing between said heparanase expressing hematopoietic cells and said heparanase non-expressing hematopoietic cells.

As used herein the phrase “heparanase expression” refers to transcription and/or translation and/or activity of heparanase. Several methods can be utilized to determine the level of heparanase expression, as is discussed hereinbelow.

The cloning and recombinant expression of the heparanase gene have been described in U.S. Pat. Nos. 5,968,822; and 6,348,344; U.S. patent applications Ser. Nos. 09/109,386; 09/258,892; 09/635,923; 09/487,716; 09/435,739; 09/776,874; 10/137,351; and 09/988,113; and in PCT Application Nos. US98/17954; US99/09256; US00/03542, which are incorporated by reference as if fully set forth herein.

Differential heparanase expression may be used as a distinguishing characteristic, evaluated at the level of gene and/or proteins expression. According to preferred embodiments of the present invention, monitoring heparanase gene expression in hematopoietic cells obtained from the subject is by monitoring heparanase RNA levels.

Monitoring Heparanase Gene Expression:

In order to isolate heparanase RNA, biological samples must be obtained for processing. Most commonly, and as preferred embodiments of the present invention, heparanized peripheral blood is drawn and RNA may be extracted from the sample, or alternatively, if desired, leukocytes may be isolated by differential gradient separation, using, for example, ficoll-hypaque or sucrose gradient solutions for cell separations, followed by ammonium chloride or hypotonic lysis of remaining contaminating erythrocytes (“Cell Biology: A Laboratory Handbook”, Volumes I-III Cellis, J. E., ed. (1994); “Current Protocols in Immunology” Volumes I-III Coligan J. E., ed. (1994); Stites et al. (eds)). Bone marrow and lymph node biopsies may be processed by collagenase/dispase treatment of the biopsy material, or by homogenization in order to obtain single cell suspensions (“Cell Biology: A Laboratory Handbook”, Volumes I-III Cellis, J. E., ed. (1994); “Current Protocols in Immunology” Volumes I-III Coligan J. E., ed. (1994); Stites et al. (eds)) whereupon RNA is subsequently isolated.

RNA may be extracted from biological samples via a number of standard techniques (see Current Protocols in Molecular Biology” Volumes I-III Ausubel, R. M., ed. (1994); Ausubel et al., “Current Protocols in Molecular Biology”, John Wiley and Sons, Baltimore, Md. (1989)). Guanidium-based methods for cell lysis enabling RNA isolation, with subsequent cesium chloride step gradients for separation of the RNA from other cellular macromolecules, followed by RNA precipitation and resuspension, is an older, less commonly employed method of RNA isolation (Glisin, Ve. Et al (1973) Biochemistry 13: 2633). Alternatively, RNA may be isolated in a single step procedure (U.S. Pat. No. 4,843,155, and Puissant, C. And Houdebine L. M. (1990) Biotechniques 8: 148-149). Single step procedures include the use of Guanidium isothiocyanate for RNA extraction, and subsequent phenol/chloroform/isoamyl alcohol extractions facilitating the separation of total RNA from other cellular proteins and DNA. Commercially available single-step formulations based on the above-cited principles may be employed, including, for example, the use of the TRIZOL reagent (Life Technologies, Gaithersburg, Md.).

According to further features of preferred embodiments of the present invention, monitoring heparanase RNA/gene expression is via a number of standard techniques well described in the art, any of which can be employed to evaluate heparanase expression. These assays comprise Northern blot and dot blot analysis, primer extension, RNase protection, RT-PCR, in-situ hybridization and chip hybridization.

Specific heparanase RNA sequences can be readily detected by hybridization of labeled probes to blotted RNA preparations extracted as above. In Northern blot analysis, fractionated RNA is subjected to denaturing agarose gel electrophoresis, which prevents RNA from assuming secondary structures that might inhibit size based separation. RNA is then transferred by capillary transfer to a nylon or nitrocellulose membrane support and may be probed with a labeled oligonucleotide probe complementary to the heparanase sequence (Alwine, et al. (1977). Proc. Natl. Acad. Sci. USA 74: 5350-5354 and Current Protocols in Molecular Biology” Volumes I-III Ausubel, R. M., ed. (1994); Ausubel et al., “Current Protocols in Molecular Biology”, John Wiley and Sons, Baltimore, Md. (1989)).

Alternatively, unfractionated RNA may be immobilized on a nylon or nitrocellulose membrane, and similarly probed for heparanase-specific expression, by Slot/Dot blot analysis. RNA slot/dot blots can be prepared by hand, or alternatively constructed using a manifold apparatus, which facilitates comparing hybridization signals by densitometry scanning (Chomczynski P. (1992) Anal. Biochem. 201: 134-139).

Primer extension is an additional means whereby quantification of the RNA may be accomplished. Primer extension provides an additional benefit in mapping the 5′ terminus of a particular RNA, by extending a primer using the enzyme reverse transcriptase. In this case, the primer is an oligonucleotide (or restriction fragment) complementary to a portion of the heparanase mRNA. The primer is end-labeled, and is allowed to hybridize to template heparanase mRNA. Once hybridized, the primer is extended by addition of reverse transcriptase, and incorporation of unlabeled dexoynucleotides to for a single-stranded DNA complementary to template heparanase mRNA. DNA is then analyzed on a sequencing gel, with the length of extended primer serving to map the 5′ position of the mRNA, and the yield of extended product reflecting the abundance of RNA in the sample (Jones et al (1985) Cell 42: 559-572 and Mierendorf R. C. And Pfeffer, D. (1987). Methods Enzymol. 152: 563-566).

RNase protection assays provide a highly sensitive means of quantifying heparanase RNA, even in low abundance. In protection assays, sequence-specific hybridization of ribonucleotide probes complementary to heparanase RNA, with high specific activity are generated, and hybridized to sample RNA. Hybridization reactions are then treated with ribonuclease to remove free probe, leaving intact fragments of annealed probe hybridized to homologous heparanase sequences in sample RNA. Fragments are then analyzed by electrophoresis on a sequencing gel, when appropriately-sized probe fragments are visualized (Zinn K. et al (1983) Cell 34: 865-879 and Melton S. A., et al (1984). Nucl. Acids Res. 12: 7035-7056).

RT-PCR is another means by which heparanase expression is verified. RT-PCR is a particularly useful method for detecting rare transcripts, or transcripts in low abundance. RT-PCR employs the use of the enzyme reverse transcriptase to prepare cDNA from RNA samples, using deoxynucleotide primers complementary to the heparanase mRNA. Once the cDNA is generated, it is amplified through the polymerase chain reaction, by the addition of dexoynucleotides and a DNA polymerase that functions at high temperatures. Through repetitive cycles of primer annealing, incorporation of dexoynucleotides facilitating cDNA extension, followed by strand denaturation, amplification of the desired sequence occurs, yielding an appropriately sized fragment that may be detected by agarose gel electrophoresis. Optimal reverse transcription, hybridization, and amplification conditions will vary depending upon the sequence composition and length(s) of the primers and target(s) employed, and the experimental method selected by the practitioner. Various guidelines may be used to select appropriate primer sequences and hybridization conditions (see, e.g., Sambrook et al., 1989, Molecular Cloning, A Laboratory Manual, (Volumes 1-3) Cold Spring Harbor Press, N.Y.; and Ausubel et al., 1989, Current Protocols in Molecular Biology, Green Publishing Associates and Wiley Interscience, N.Y.).

In-situ hybridization provides a powerful and versatile tool for the detection and localization of cell/tissue specific heparanase RNA expression. Labeled anti-sense RNA probes are hybridized to mRNAs in cells singly, or in processed tissue slices, which are immobilized on microscope glass slides (In Situ Hybridization: Medical Applications (eds. G. R. Coulton and J. de Belleroche), Kluwer Academic Publishers, Boston (1992); In Situ Hybridization: In Neurobiology; Advances in Methodology (eds. J. H. Eberwine, K. L. Valentino, and J. D. Barchas), Oxford University Press Inc., England (1994); and In Situ Hybridization: A Practical Approach (ed. D. G. Wilkinson), Oxford University Press Inc., England (1992)). Numerous non-isotopic systems have been developed to visualize labeled DNA probes including; a) fluorescence-based direct detection methods, b) the use of digoxigenin- and biotin-labeled DNA probes coupled with fluorescence detection methods, and c) the use of digoxigenin-and biotin-labeled DNA probes coupled with antibody-enzyme detection methods. When fluorescence-labeled anti-sense RNA probes are hybridized to cellular RNA, the hybridized probes can be viewed directly using a fluorescence microscope. Direct fluorochrome-labeling of the nucleic acid probes eliminate the need for multi-layer detection procedures (e.g., antibody-based-systems), which allows fast processing and also reduces non-specific background signals, hence providing a versatile and highly sensitive means of identifying heparanase gene expression.

Chip hybridization utilizes heparanase specific oligonucleotides attached to a solid substrate, which may consist of a particulate solid phase such as nylon filters, glass slides or silicon chips [Schena et al. (1995) Science 270:467-470] designed as a microarray. Microarrays are known in the art and consist of a surface to which probes that correspond in sequence to gene products (such as cDNAs) can be specifically hybridized or bound at a known position for the detection of heparanase gene expression.

Quantification of the hybridization complexes is well known in the art and may be achieved by any one of several approaches. These approaches are generally based on the detection of a label or marker, such as any radioactive, fluorescent, biological or enzymatic tags or labels of standard use in the art. A label can be applied to either the oligonucleotide probes or the RNA derived from the biological sample.

In general, mRNA quantification is preferably effected alongside a calibration curve so as to enable accurate mRNA determination. Furthermore, quantifying transcript(s) originating from a biological sample is preferably effected by comparison to a normal sample, which sample is characterized by normal expression pattern of the examined transcript(s).

Heparanase expression may also be evaluated at the level of protein expression, either by demonstration of the presence of the protein, or by its activity, with activity herein referring to the catalytic endoglycosidase activity of the heparanase enzyme.

Monitoring Heparanase Protein Expression:

Anti heparanase antibodies for use in heparanase-specific protein detection are described in U.S. Pat. No. 6,177,545; U.S. patent application Ser. Nos. 09/704,772; 09/322,977; 09/186,200; 09/944,602; 09/759,207; and PCT Application Nos. US99/25451 and US99/09255, which are incorporated by reference as if fully set forth herein. The antibodies bind both native and denatured heparanase protein and may be detected by several well-known assays in the art, including ELISA, RIA, light emission immunoassays, Western blot analysis, immunofluorescence assays, immunohistochemistry and FACS analysis.

Enzyme linked immunosorbant (ELISA) assays and radioimmunoassays (RIA) follow similar principles for detection of specific antigens, in this case, heparanase. In RIA a heparanase-specific antibody is radioactively labeled, typically with ¹²⁵I. In ELISA assays a heparanase-specific antibody is chemically linked to an enzyme. Heparanase specific capturing antibody is immobilized onto a solid support. Unlabelled specimens, e.g., protein extracts from biopsy or blood samples are then incubated with the immobilized antibody under conditions where non-specific binding is blocked, and unbound antibody and/or protein removed by washing. Bound heparanase is detected by a second heparanase specific labeled antibody. Antibody binding is measured directly in RIA by measuring radioactivity, while in ELISA binding is detected by a reaction converting a colorless substrate into a colored reaction product, as a function of linked-enzyme activity. Changes can thus readily be detected by spectrophotometry (Janeway C. A. et al (1997). “Immunbiology” 3^{rd} Edition, Current Biology Ltd., Garland Publishing Inc.; “Cell Biology: A Laboratory Handbook”, Volumes I-III Cellis, J. E., ed. (1994); “Current Protocols in Immunology” Volumes I-III Coligan J. E., ed. (1994); Stites et al. (eds)). Both assays therefore provide a means of quantification of heparanase protein content in a biological sample.

Heparanase protein expression may also be detected via light emission immunoassays. Much like ELISA and RIA, in light emission immunoassays the biological sample/protein extract to be tested is immobilized on a solid support, and probed with a specific label, labeled anti-heparanase antibody. The label, in turn, is luminescent, and emits light upon binding, as an indication of specific recognition. Luminescent labels include substances that emit light upon activation by electromagnetic radiation, electro chemical excitation, or chemical activation and may include fluorescent and phosphorescent substances, scintillators, and chemiluminescent substances. The label can be a part of a catalytic reaction system such as enzymes, enzyme fragments, enzyme substrates, enzyme inhibitors, coenzymes, or catalysts; part of a chromogen system such as fluorophores, dyes, chemiluminescers, luminescers, or sensitizers; a dispersible particle that can be non-magnetic or magnetic, a solid support, a liposome, a ligand, a receptor, a hapten radioactive isotope, and so forth (U.S. Pat. Nos. 6,410,696, U.S. Pat. No. 4,652,533 and European Patent Application No. 0,345,776), and provide an additional, highly sensitive method for detection of heparanase protein expression.

Western blot analysis is another means of assessing heparanase protein content in a biological sample. Protein extracts from biological samples of hematopoietic cells are solubilized in a denaturing ionizing environment, and aliquots are applied to polyacrylamide gel matrixes. Proteins separate based on molecular size properties as they migrate toward the anode. Antigens are then transferred to nitrocellulose, PVDF or nylon membranes, followed by membrane blocking to minimize non-specific binding. Membranes are probed with antibodies directly coupled to a detectable moiety, or are subsequently probed with a secondary antibody containing the detectable moiety. Typically the enzymes horseradish peroxidase or alkaline phosphatase are coupled to the antibodies, and chromogenic or luminescent substrates are used to visualize activity (Harlow E. et al (1998) Immunoblotting. In Antibodies: A Laboratory Manual, pp. 471-510 CSH Laboratory, cold Spring Harbor, N.Y. and Bronstein I. Et al. (1992) Biotechniques 12: 748-753).

Unlike RIA, ELISA, light emission immunoassays and immunblotting, which quantify heparanase protein content in whole samples, immunofluorescence/immunocytochemistry may be used to detect proteins in a cell-specific manner, though quantification is compromised.

Hematopoietic cells may be isolated by methods listed hereinabove, and cell suspensions may be used to prepare cytospin preparations for analysis. Alternatively, blood smears may be prepared via standard hematological processes. Once cells are deposited on slides, they may be fixed, and probed with labeled antibody for detection of heparanase in a cell specific fashion. Antibodies may be directly conjugated to fluorescent markers, including fluorescein, FITC, rhodamine, Texas Red, Cy3, Cy5, Cy7, and other fluorescent markers, and viewed in a fluorescent microscope, equipped with the appropriate filters. Antibodies may also be conjugated to enzymes, which upon addition of an appropriate substrate commence a reaction providing a colored precipitate over cells with detected heparanase protein. Slide may then be viewed by standard light microscopy. Alternatively, primary antibodies specific for heparanase may be further bound to secondary antibodies conjugated to the detectable moieties. Cell surface expression can be thus assessed, and the addition of cell permeabilization solutions, such as Triton-X and saponin may be applied to facilitate reagent penetration within cell cytoplasms (“Cell Biology: A Laboratory Handbook”, Volumes 1-111 Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980)).

Immunohistochemistry is quite similar to immunofluorescence or immunocytochemistry, in principle, however tissue specimens are probed with heparanase antibody, as opposed to cell suspensions. Biopsy specimens are fixed and processed and optionally embedded in paraffin, sectioned if needed, providing cell or tissue slides subsequently probed with heparanase specific antibodies. Alternatively, frozen tissue may be sectioned on a cryostat, with subsequent antibody probing, obviating fixation-induced antigen masking. Antibodies, as in immunofluorescence or immunocytochemistry, are coupled to a detectable moiety, either fluorescent, or enzyme-linked, and are used to probe tissue sections by methods described for immunofluorescence, and are subsequently visualized by fluorescent or confocal microscopy, depending upon the detection method employed. Visualization of a reaction product precipitate may be viewed by standard light microscopy, if an enzymatic detectable moiety was utilized, following development of the reaction product (“Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980)).

Fluorescent activated cell sorting (FACS) analysis is another means by which to assess heparanase expression. A general description of FACS apparatus and methods in provided in U.S. Pat. Nos. 4,172,227; 4,347,935; 4,661,913; 4,667,830; 5,093,234; 5,094,940; and 5,144,224, incorporated herein by reference. Cells are introduced into the FACS machine and are delivered via tubing into the FACS cell, which they pass through as single cells. A laser beam is directed at the FACS cell, and forward laser scatter is collected by a photodiode, side laser scatter is directed to a PMT tube via a lens, directed to PMT1. Specific filters direct fluorescence from the side scatter to other PMT tubes for multivariate analysis. Side laser scatter is a reflection of cell size and granularity, and may be used to identify cell populations in mixed samples. Cells labeled with fluorescent anti-heparanase antibody may be detected by laser excitation and collection via PMT tubes, which can be identified for cell type via size and granularity, or via incorporation of additional cell surface markers for identification. Typically, FACS analysis is used for determination of cell surface expression of a particular protein, and hence heparanase specific antibodies may be utilized for probing detection of cell surface heparanase expression in hematopoietic cell populations. Specific hematopoietic cell subtypes expressing surface heparanase protein may be ascertained by size and granularity characteristics, or alternatively by co-staining with additional cell surface marker proteins.

Monitoring Heparanase Protein Activity

Demonstration of the absence or presence of heparanase activity within a sample is an additional means of distinguishing heparanase expressing versus non-expressing populations. Heparanase catalytic activity assays are described in the Patents and Applications listed above and further in U.S. Pat. No. 6,190,875; U.S. patent application Ser. No. 09/753,692 and PCT Application No. US99/15643, which are incorporated by reference as if fully set forth herein. In principle, heparanase activity is measured in the presence of a heparanase substrate, wherein cleavage of the substrate into specific products serves as an indicator for enzyme activity.

According to features of preferred embodiments of the present invention, the heparanase substrates may be selected from the group consisting of soluble or immobilized heparan sulfate proteoglycans, heparan sulfate or heparin. Further features of preferred embodiments of the present invention include the incorporation of a detectable moiety within the assay, to effect ease of identification of heparanase activity. Detectable moieties used as such may be selected from the group consisting of chromogenic moieties, fluorogenic moieties, radioactive moieties and light-emitting moieties, enabling quantitative evaluation of heparanase activity via a suitable detecting equipment, e.g., a spectrophotometer, fluorometer or luminometer, β-emission counter, a densitometer, and others. A preferred quantitative calorimetric assay is the tetrazolium blue (an oxidative reagent) assay in which the reagents are reduced to a soluble colored formazan salt by the degraded substrate.

Hematopoietic cell heparanase expression may be determined in cells derived from the bone marrow, the lymph node, or peripheral blood.

Thus heparanase expression may be determined in hematopoietic cell populations, whereupon heparanase expressing versus heparanase non-expressing cells may be readily determined.

Accordingly, and representing preferred embodiments of the present invention, normal heparanase expressing hematopoietic cells may comprise activated T or B lymphocytes, platelets, granulocytes, macrophages, monocytes, mast cells, megakaryocytes, promegakaryocytes, megakaryoblasts, promyelocytes, metamyelocytes, myelocytes, myeloblasts, monoblasts, and dendritic cells.

Normal heparanase non-expressing hematopoietic cells may, in turn, comprise erythrocytes, normoblasts, erythroblasts hematopoietic stem cells and naive T and B lymphocytes.

While heparanase expression in hematopoietic cell populations may be determined individually, as a means of classifying expressing versus non-expressing cells, it is also to be envisioned that numerous other marker proteins may be concurrently evaluated, in order to further classify hematopoietic cell populations.

Thus according to yet another preferred embodiment of the present invention hematopoietic cells monitored for heparanase expression may additionally express the surface markers CD3, CD14 and/or CD15, and others.

It should be noted that further description of methodology, which can be used to determine heparanase expression levels, is provided in the Examples section, which follows.

The results described in the Examples section emphasize the feasibility of using heparanase expression to distinguish between various normal and neoplastic hematopoietic cell populations.

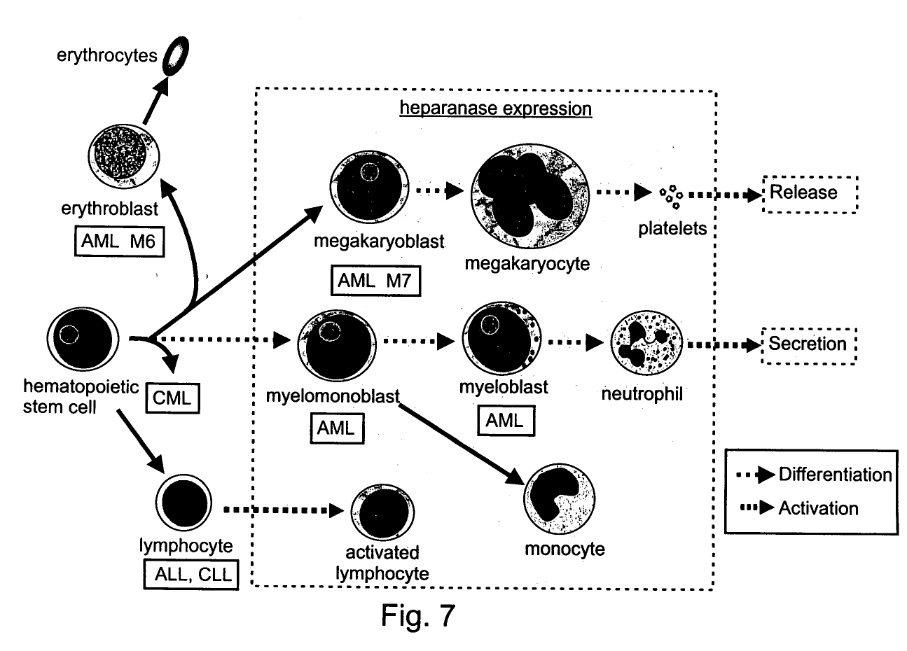

Heparanase RNA expression was found in all samples of acute myelogenous leukemia evaluated, except for erythroleukemia. Neither the erythroblastic cell line (K562) nor mature erythrocytes revealed heparanase gene expression, indicating a lack of heparanase expression in the erythroid lineage (FIG. 7). Heparanase expression was detected in AML cells of both early and late stages of myeloid differentiation (M1, M2, M3, M4, M5 and M7), as well as in mature circulating granulocytes. These data indicate an early heparanase gene expression checkpoint within the myeloid differentiation pathway (FIG. 7). Heparanase expression was therefore, found to be sustained throughout the differentiation of the myeloid lineage (FIG. 7).

While ample heparanase expression was detected in early myeloid cells, heparanase RNA, intracellular and cell-surface protein were not detected in most CML samples evaluated (Table 1 and Example 2). CML is a multipotential stem cell disease affecting the myeloid lineage. It was previously shown that CML cells lack certain enzymatic activities (e.g., leukocyte alkaline phosphatase) which are associated with late stages of myeloid differentiation (24, 25). The fact that heparanase RNA and protein were detected in relatively less differentiated AML cells, yet were absent in CML indicates that CML originates from a very early myeloid stem cell, prior to the differentiation-related activation of heparanase gene expression (FIG. 7).

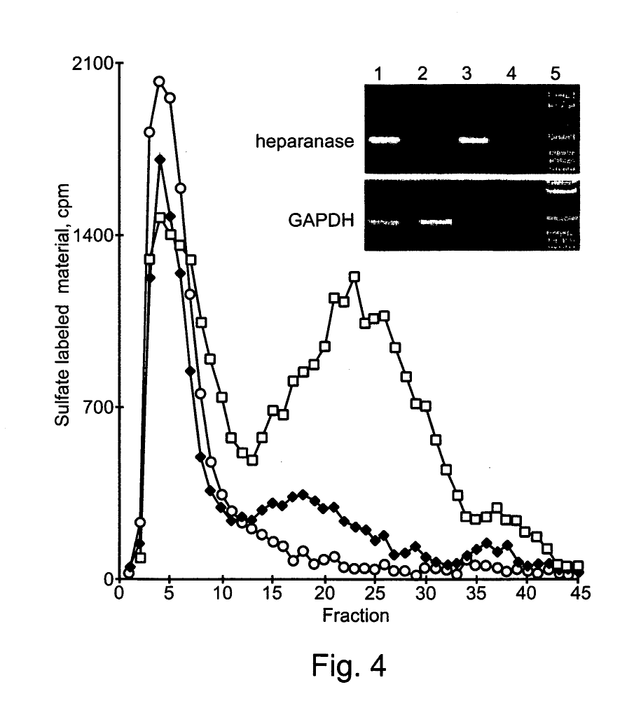

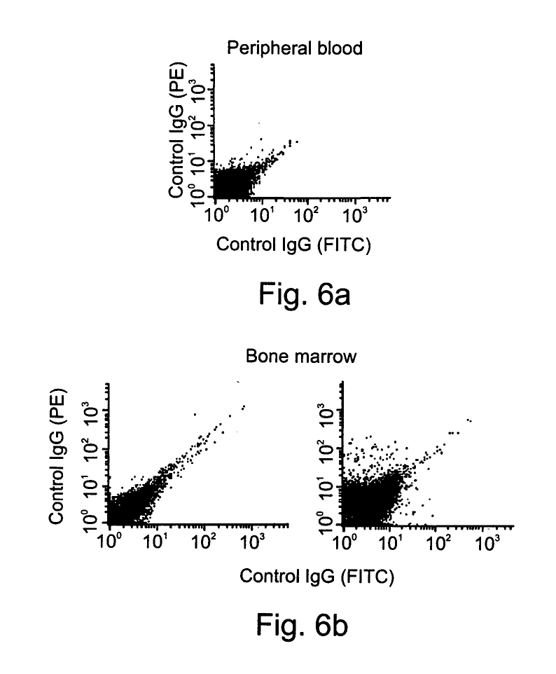

No heparanase expression was detected in normal or aberrant circulating lymphoid cells (FIG. 3B and FIG. 5B), or in CD34⁺ mobilized progenitor cells. The lack of heparanase expression in early hematopoietic progenitors, lymphoid cells and most leukemic cell types indicates that these cells do not depend on heparanase activity for transmigration from the bone marrow into peripheral circulation. Cellular trafficking from bone marrow to peripheral circulation is therefore more likely controlled by other cellular proteins, (such as integrins and the cytoskeletal proteins), and not by ECM-degrading enzymes. CML transmigration from bone marrow to peripheral circulation has been attributed to a loss of normal cytoskeletal/integrin function in response to BCR/Abl oncogene activity (26, 27), and it is therefore consistent that heparanase expression was absent in most of the CML samples.

Thus, heparanase expression serves as an indicator for hematopoietic cell subtypes, and leukemic cell origins, and as such provides numerous, critical diagnostic applications.

For convenience, a number of terms and their definitions will be grouped here:

As used herein the phrase “Acute Lymphocytic Leukemia (ALL)” refers to a disease in which cells originating in the bone marrow that normally develop into lymphocytes become cancerous and rapidly replace normal cells in the bone marrow.

As used herein the phrase “Chronic Lymphocytic Leukemia (CLL)” refers to a disease characterized by a large number of cancerous mature lymphocytes originating in the bone marrow.

As used herein the phrase “Actue Myelogenous Leukemia (AML)” refers to a disease in which myelocytes originating in the bone marrow become cancerous and rapidly replace normal cells in the bone marrow.

As used herein the phrase “Chronic Myelogenous Leukemia (CML)” refers to a disease in which neoplastic cells of myeloid origin proliferate in the bone marrow.

As used herein the phrase “Non-Hodgkin's Lymphoma (NHL)” refers to a group of related malignancies that originate in the lymphatic system, and usually spread throughout the body.

As used herein the phrase “undifferentiated myelocytic cells” refers to neoplastic cells of myeloid origin expressing a phenotype of early undifferentiated cells, characterized to date by the presence of azurophic granules, an enlarged diameter, and surface marker expression of CD13, CD34, CD33, and the absence of differentiation markers CD11b, CD14, CD15.

As used herein the phrase “differentiated B lymphoma cells” refers to lymphatic system neoplastic cells of lymphocyte origin expressing a differentiated phenotype, characterized to date by the presence of at least the B220 pan B cell marker and optionally other surface markers.

As used herein the phrase “B leukemia cells” refers to bone marrow, neoplastic cells of lymphocyte origin expressing a differentiated phenotype, characterized to date by the presence of the B220 pan B cell marker.

Accordingly, as a diagnostically beneficial protocol, and as part of preferred embodiments of the present invention, the method of monitoring heparanase expression as delineated hereinabove is used to distinguish between heparanase expressing myeloid cells (AML) and heparanase non-expressing myeloid cells (CML), differentiated B lymphoma cells and/or between heparanase non-expressing B leukemia cells.

According to still further features of preferred embodiments, the method may be used to distinguish between heparanase expressing acute myelogenous leukemia cells and heparanase non-expressing chronic or acute lymphocytic leukemia cells, chronic myelogenous leukemia cells and/or heparanase non-expressing non-Hodgkin's lymphoma cells.

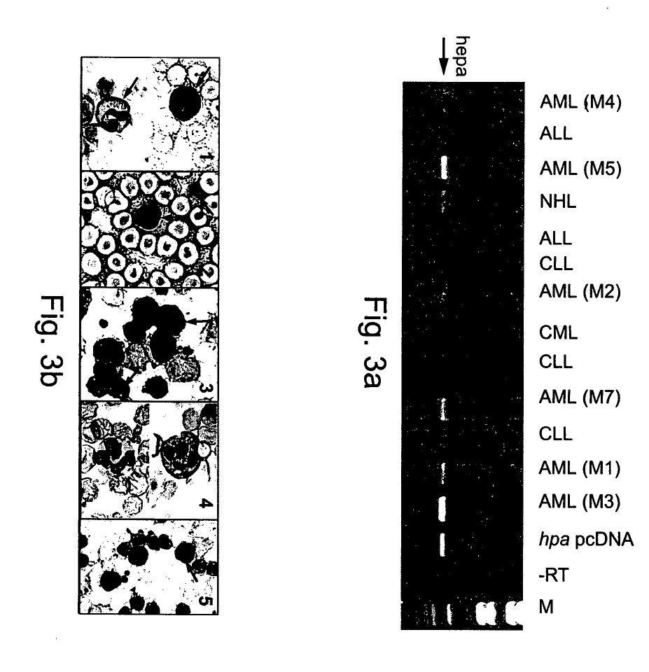

Heparanase expression has focused on cytoplasmic localization of the heparanase protein, as has been demonstrated in the literature (29, 30), and herein, in FIGS. 2 and 3, and as described in Examples 1 and 2 of the Examples section that follows. The amino acid sequence of human heparanase, however, contains a stretch of C-terminal hydrophobic amino acids, and complete solubilization of the enzyme requires detergent extraction, implying its potential association with the plasma membrane (13, 14). It was suggested that in highly malignant breast and bladder carcinoma cells, cytoplasmic enzymes, such as cathepsin B, are translocated to the cell-surface (31, 32). Neutrophil heparanase co-localizes with gelatinase in tertiary granules (29, 30) within the cytoplasm of the cell; however, minimal cell surface expression of heparanase was detected in normal hematopoietic cells.

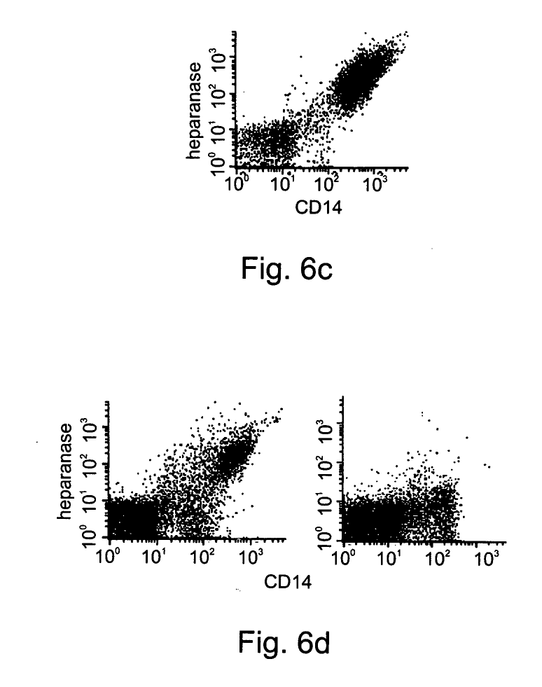

It was suggested that in highly malignant breast and bladder carcinoma cells, cytoplasmic enzymes such as cathepsin B are translocated to the cell-surface (31, 32). Indeed a most unexpected finding of the present invention was that heparanase hematopoietic cell surface expression was significantly higher in some AML patients, surpassing that of normal hematopoietic cells, and absent in other neoplastic hematopoietic cells (FIGS. 5 and 6). Most unexpected was the finding that expression declined corresponding to clinical signs of patient remission, supporting a definitive role for surface heparanase expression in AML malignancy.

It appears therefore that in some malignancies including breast and bladder carcinomas (31, 32), and certain types of leukemias (as demonstrated in the present study), enzymes normally stored within cytoplasmic granules can translocate to the cell-surface, and as such can be exploited for use as neoplastic markers. Additionally, and concurrently, evaluation of cytoplasmic expression of heparanase in specific hematopoietic neoplastic cells provides a diagnostically useful tool in distinguishing between normal and specific hematopoietic neoplastic cells.

Accordingly, it is another object of the present invention to provide an improvement in a process of diagnosing acute myelogenous leukemia, versus non-Hodgkin's lymphoma, chronic or acute lymphocytic leukemia or chronic myelogenous leukemia, the improvement comprising determining whether hematopoietic cells suspected as neoplastic and in particular, leukemic cells express heparanase, wherein heparanase expression is indicative of acute myelogenous leukemia and wherein non-expression of heparanse in neoplastic hematopoietic cells is indicative of non-Hodgkin's lymphoma or chronic or acute lymphocytic leukemia or chronic myelogenous leukemia.

Current methods for diagnosing and differentiating between leukemias and lymphomas entail a series of tests and data collected from physical exams. Bone marrow or lymph node biopsies and analysis of peripheral blood samples for cytogenetic and/or immunologic analysis is standard practice. Biopsy material is typically processed and embedded in paraffin, for examination of histo-pathologic changes, and immunostaining for determinative markers for staging and typing of the different malignancies is conducted. Frozen tissue specimens may be obtained as well, and immunostained for cancer-specific markers.

If morphological, immunophenotypic, and cytogenetic techniques are employed, classification of various subgroups of AML and ALL can be accomplished. Employing this approach is time-consuming, costly, and provides opportunity for error. Immunophenotypic classification, for example, typically entails evaluation of numerous markers, including: CD13, CD33, CD34, HLA-DR, CD11b, CD14, CD41, CD42a, CD42b, CD61 and TdT expression, however some of these markers are expressed on both ALL and AML, confounding accurate diagnosis. For example, TdT staining typically positive in ALL and negative in AML be found in the reverse, therefore, is not very useful in distinguishing between these 2 types of acute leukemias. Furthermore, though patients with AML demonstrate myeloid markers such as CD33, whereas patients with ALL demonstrate lymphoid markers, ALL cases can aberrantly express myeloid markers, such as CD13 (Preti H A, Huh Y O, O'Brien S M. Cancer (1995) 76(9): 1564-70; Ludwig W D, Rieder H, Bartram C R. Blood (1998) 92(6): 1898-909).

The differential diagnosis of CLL as well, is confounded by several other entities that share common surface marker phenotype, such as hairy cell leukemia, which is moderately positive for surface membrane immunoglobulins of multiple heavy-chain classes and typically negative for CD5 and CD21, similar to what is found for CLL of B cell origin. The pattern of positivity for CLL of T cell origin, positive staining for CD19, CD20, and the T-cell antigen CD5 is shared by mantle cell lymphoma, as well, further complicating differential diagnosis, much as positivity for β₂-microglobulin is shared between CLL and NHL. CML shares some markers in common with CLL in immunophenotypic classification, and despite evidence of the presence of the characteristic Philadelphia chromosome that can confirm CML, however, many CML do not reveal evidence of the Philadelphia chromosome (Rai KR, Keating M. Cancer Medicine 1997; 2697-728. Rai KR, Sawitsky A, Cronkite EP. Blood 1975 46(2): 219-34).

Thus, the current state of the art does not provide a single marker that can be used to effectively differentiate between the various types of leukemias and lymphomas. Further, the current state of the art requires multi-parameter analysis to provide a diagnosis, which may still prove inaccurate in typing and staging hematopoietic cell neoplasms. Clearly a single marker for differential diagnosis is lacking, and of critical importance, though even a single method of analysis offers numerous diagnostic advantages.

Heparanase expression and activity in all CLL and most other types of leukemias was not detected (Table I and FIG. 3), nor does it characterize other hematological disorders (Bitan M., Exp. Hematol. 2002 January; 30(1):34-41) Therefore, heparanase expression, at the RNA, protein or activity levels, may serve as an independent marker applied to support the identification of AML and to distinguish this class of leukemias from CML or leukemias of the lymphoid lineage. In addition, the lack of heparanase expression may help to identify circulating CML cells independently of other common but rather problematic techniques (e.g., alkaline phosphatase staining, 33). Other enzymes (e.g., metalloproteinases) comprise multi-gene families with variable degrees of sequence homologies and structural similarities, making their usage in diagnosis difficult. In contrast, only one heparanase gene has been detected to date, facilitating the development of efficient and reliable molecular diagnostic methodology, with a broad range in host applicability.

An additional advantage in the diagnosis and treatment of leukemia would be an effective means to ex-vivo assess a potential anti-neoplastic drug. As chemotherapy is often toxic to the host, and tumor cell responsiveness to chemotherapy is often unpredictable, a screening method for drug efficacy would be a highly beneficial, cost-effective and a potentially life-saving procedure.

Therefore, according to still another aspect of the present invention, there is provided a method for ascertaining the suitability of an anti-neoplastic drug candidate for efficacy in treating acute myelogenous leukemia, wherein the method comprises combining the candidate drug with a cell having an acute myelogenous leukemia leukemia phenotype, the cell being isolated or in a biological sample and detecting in the cell or biological sample a change in heparanase expression.

Methods for the evaluation of heparanase expression may be optimized to provide efficient and quality-controlled assays for applications in a clinical setting. Hence the construction of optimized kits containing reagents for the rapid detection of heparanase expression and instructions to facilitate identification and quantification of heparanase expression at either the RNA or protein level fulfills an important niche in the clinical diagnosis of leukemia and lymphoma.

It is therefore still another object of the present invention to provide kits for distinguishing between heparanase expressing acute myeloid leukemic cells, other neoplastic and/or normal hematopoietic cells and heparanase non-expressing non-Hodgkin's lymphoma cells, chronic or acute lymphocytic leukemia cells, chronic myeloid leukemic cells, other neoplastic and/or normal hematopoietic cells, comprising at least one container including at least one reagent for determining a level of heparanase expression or activity in a biological sample, and packaging material identifying the reagent for use in distinguishing between one or more of the above listed heparanase expressing cells and one or more of the above listed heparanase non-expressing neoplastic or normal hematopoietic cells.

It is to be appreciated that heparanase expression may be evaluated as disclosed hereinabove, at the RNA level, at the protein level or at the enzymatic activity level, and as such, represents additional preferred embodiments of the present invention. Kit construction includes the provision of at least one reagent for the detection of heparanase RNA, heparanase protein, and/or heparanse protein activity, via standard assays and methods discussed hereinabove, and as well known in the art. Reagents may be designed to incorporate a detectable moiety as well, as outlined hereinabove, and hence the design of reagents specifically interacting with a heparanase product, either RNA or protein, providing a detectable indicator of heparanase expression or its activity within a given sample, as outlined by methods detailed hereinabove, all represent additional preferred embodiments of the present invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Heparanase Localization in Normal Peripheral Blood Granulocytes and Monocytes

### Material and Experimental Methods

Obtaining Antibodies

The mouse anti-heparanase monoclonal antibody (mAb) 130, an IgG1 subclass, was generated as previously described (13, U.S. Pat. No. 6,177,545; U.S. patent application Ser. Nos. 09/704,772; 09/322,977; 09/186,200; 09/944,602; 09/759,207; and PCT Application Nos. US99/25451 and US99/09255). FITC-labeled anti-heparanase mAb 130 was prepared as follows; 2 mg of antibody were disloved in 1 ml of 0.1 M Sodium Carbonate, pH 9.0. 50 μl of 1 mg/ml fresh FITC solution in DMSO were added to 1 ml antibody solution, by 5 μl increments. The mixture was incubated for 8 hours at 4° C. in the dark. When OD495 nm/280 nm in the range of 0.3 to 1.0, the reaction was stoped by adding NH₄Cl to a final concentration of 50 mM and incubateed for 2 hours at 4° C. Xylene cylanol was then added to a final concentration of 0.1% and glycerol to a final concentration of 5%. One ml of the resulting mixture was loaded on 20 ml column, fractions were collected and tested using the Bradford method. FITC-conjugated anti CD3, CD14, CD15, CD19 and CD34 mAbs and isotype-matched (IgG1) control mAb were purchased form Dako (Glostrup, Denmark) and utilized according to manufacturer instructions. FITC and PE-conjugated goat anti mouse antibodies were purchased from Dako and Jackson ImmunoResearch Laboratories (West Grove, Pa.), respectively.

Blood Samples

Peripheral blood and bone marrow samples were obtained from discarded material utilized for routine laboratory testing at the Hematology Laboratory, Tel-Aviv Sourasky Medical Center, Tel-Aviv, and the Hematology Department, Hadassah-Hebrew University Hospital, Jerusalem. Informed consent was obtained from all patients.

RNA Isolation and RT-PCR

Total RNA was isolated utilizing the TRIzol™ reagent (Life Technologies, Gaithersburg, Md.) according to manufacturer's instructions and subjected to RT-PCR as previously described (13). Briefly, Reverse transcription of 500 ng total RNA from normal and leukemia mononuclear cells samples was performed by oligo (dT) priming, followed by PCR amplification, utilizing the following oligonucleotide primers: HPU-355 (5′-TTCGATCCCAAGAAGGAATCAAC-3′; SEQ ID NO:1) and HPL-229 (5′-GTAGTGATGCCATGTAACTGAATC-3′; SEQ ID NO:2). Aliquots (10 μl) of the amplification products were resolved by 1.5% agarose gel electrophoresis and visualized with ethidium bromide staining. Only RNA samples that failed to amplify via PCR, in the absence of reverse transcriptase were further analyzed. The following conditions were applied for PCR amplification of heparanase mRNA: 94° C. incubation for 4 minutes, followed by 29 cycles of incubation at 94° C. for 45 seconds followed by incubation at 60° C. for 1 minute and finally incubation at 72° C. for 45 seconds. Final amplified PCR products were then incubated at 4° C. for 1 hour. The total numbers of samples evaluated for heparanase RNA expression were: chronic lymphocytic leukemia (CLL): 33; acute lymphocytic leukemia (ALL): 8; acute myelogenous leukemia (AML): 15; chronic myelogenous leukemia (CML): 8; non-Hodgkin's lymphoma (NHL): 7 and normal donors: 8.

Isolation of Peripheral Blood Monocytes

Mononuclear cells were isolated from normal donor peripheral blood by Ficoll-Hypaque (Pharmacia, Uppsala, Sweden) density gradient centrifugation, and washed 2 times in phosphate buffered saline (PBS). Adherent cells were collected from the mononuclear cell fraction by incubation at 37° C. for 2 hours in RPMI on plastic tissue culture flasks. FACS analysis of the adherent monocyte cell preparation was performed using monoclonal antibodies probing for the presence of the cell surface markers CD14 and CD45, which are markers specific for monocyte populations. Less than 5% of the cells were CD14⁻/CD45⁺.

Immunofluorescence Staining of Heparanase in Normal Circulating Human Granulocytes

Granulocyte-enriched fractions were purified from normal blood samples by Ficoll-Hypaque density gradient centrifugation and washed twice with PBS. Immunofluorescence staining with anti-heparanase mAb 130 was then performed on solubilized cells. For this purpose, polymorphonuclear enriched fractions were applied on glass slides, and then permeabilized with 0.5% Triton X-100 followed by immunofluorescent staining with mAb 130 as previously described (18).

Immunocytochemistry

Cytospins prepared from peripheral blood cells were fixed in acetone:methanol (1:1) for 10 minutes at 24° C. Slides were washed in PBS and endogenous peroxidases were blocked with 0.3% H₂O₂ in methanol for 15 minutes at room temperature. The slides were then incubated for an additional 15 minutes at 24° C. with PBS containing 10 mM glycine and 10 μg/ml BSA, followed by blocking for an additional 30 minutes with normal 5% goat serum in PBS containing 1% BSA.

Slides were then incubated with a range of dilutions (1:50-1:200 dilutions) of affinity-purified monoclonal anti-heparanase mAb 130, for 4 hours at 4° C., followed by incubation for 1 hour at room temperature with biotinylated secondary goat-anti-mouse IgG antibodies and then an additional 1 hour incubation with a streptavidin-peroxidase conjugate (1:200 dilution) (Histostain-SP™, Zymed Laboratories Ltd.), resulting in the deposition of a precipitate, which enabled visualization of the reaction product, and hence marked the site of heparanase localization (14, 19).

### Experimental Results

Heparanase Expression in Normal Circulating Leukocytes

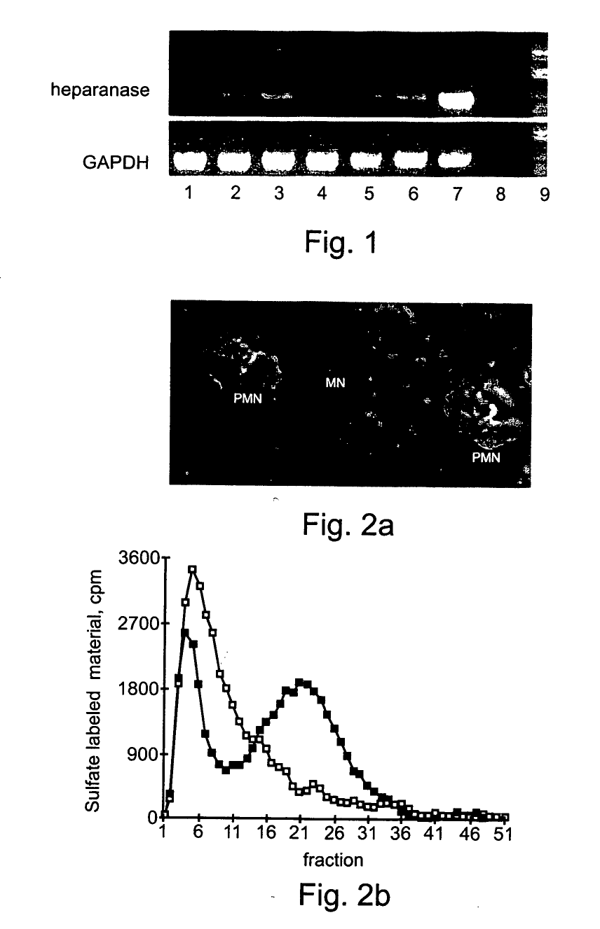

Heparanase mRNA expression was analyzed in normal peripheral blood leukocytes. RNA was extracted from samples of peripheral blood leukocytes (n==8) and semi-quantitative RT-PCR was then performed, utilizing previously described oligonucleotide primers (14). As expected, a PCR product yielding a 585 bp single band was detected (FIG. 1). Relatively high levels of heparanase mRNA were found in all samples derived from normal donors of peripheral blood leukocytes (FIG. 1, lanes 1-6, two additional positive samples are not shown).

RT-PCR analysis of heparanase mRNA expression provided information regarding organ expression of the gene product, however cell specific heparanase expression was lacking, due to the heterogeneity of the peripheral leukocyte population comprising the sample utilized for RNA extraction. Therefore, heparanase cellular localization in hematopoietic cells was verified by immunofluorescence staining. Enriched polymorphonuclear cell populations (granulocytes) probed with the monoclonal antibody (mAb) 130 revealed ample presence of heparanase in cell cytoplasms (FIG. 2A). In contrast, heparanase was not detected in mononuclear cells (lymphocytes) or erythrocytes (FIG. 2A). In order to examine whether monocytes expressed heparanase, peripheral adherent monocytes were isolated. As shown in FIG. 2B, profound level of heparanase activity was detected in the monocytes, which was inhibited by heparin. Heparanase mRNA expression was detected in isolated monocytes by RT-PCR, as well (data not shown). These results indicated that most of the heparanase protein present in circulating mature leukocytes was localized within the cytoplasms of granulocytes and monocytes.

### Example 2

### Heparanase Localization in Myeloblasts of Leukemic Patient Samples

### Material and Experimental Methods

Blood Samples, RNA Isolation, RT-PCR, Isolation of Peripheral Blood Monocytes and Immunocytochemistry

These procedures were conducted as per Example 1 above.

### Experimental Results

Heparanase mRNA and Protein Expression in Leukemic Cells

Aberrant expression of ECM-degrading metalloproteinases may characterize specific types of malignant hematopoietic cells (8). Heparanase expression was therefor evaluated both at the mRNA and protein levels in samples obtained from patients with a variety of hematological disorders, most predominantly with leukemias. RT-PCR amplification of mononuclear heparanase mRNA expression was evaluated in mononuclear of patient mRNA samples (FIG. 3A). Surprisingly, heparanase mRNA expression was detected primarily in AML samples. CLL samples failed to provide a positive signal for heparanase mRNA, regardless of CLL patient clinical staging. Heparanase mRNA was detected in 14 AML samples classified as M2, M3, M4, M5 and M7. Heparanase expression was not detected, however, in a single case of AML classified as M6 (erythroleukemia). Similarly, heparanase expression and activity was not detected in the K562 erythroleukemia cell line (not shown).

Cumulatively, (Table 1) the results indicate no heparanase mRNA detected in any of the CLL (0 out of 33) or NHL (0 out of 7) samples examined. Only 2 out of 8 (25%) ALL were positive for heparanase mRNA expression. The six heparanase negative samples were newly diagnosed patients, while the two heparanase positive ALL samples were from patients suffering clinical relapse. Eight CML samples were examined for heparanase expression, all from patients in the chronic phase, all positive for BCR/Abl oncogene expression. Of these, only 1 out of 8 CML samples was positive for heparanase mRNA expression.

TABLE 1
Expression of heparanase mRNA in human leukemias and lymphomas

| | Number of | Number of | Number of |
|---|---|---|---|
| Type | patients | Heparanase⁺ | Heparanase⁻ |
| CLL | 33 | 0 | 33 |
| AML | 15 | 14 | 1 |
| ALL | 8 | 2 | 6 |
| CML | 8 | 1 | 7 |
| NHL | 7 | 0 | 7 |
| Cord blood | 14 | 1 | 13 |

These data were confirmed by immunodetection of heparanase protein in leukocyte populations (FIG. 3B, reddish-brown staining), in particular within the cytoplasm of AML blasts (FIG. 3B, panel 5). Similar staining was seen within normal granulocytes and monocytes (FIG. 2 and FIG. 3B panel 1, respectively). In contrast, no heparanase staining was detected in CLL (FIG. 3B panel 3), or CML (FIG. 3B panel 4) cells, nor in normal circulating lymphocytes (FIG. 3B panel 2).

### Example 3

### Functional Heparanase is Exclusive to AML Patient Leukocytes

### Material and Experimental Methods

Blood Samples

Peripheral blood samples were obtained from AML stage 4 and CLL patients.

Isolation of Peripheral Blood Leukocytes,

Platelet depleted preparations of peripheral blood leukocytes were isolated by Ficoll-Hypaque density gradient centrifugation, prepared from AML stage M4 and CLL patients, as described in Example 1 above, and in (9-11, 14).

RNA Isolation, RT-PCR:

These procedures were conducted as detailed in Example 1, above.

Heparanase Activity

Dishes coated with metabolically labeled extracellular matrix (ECM) were prepared as previously described (9-11, 14). AML-M4 and CLL patient leukocytes were suspended (2.5×10⁶/ml) in serum free RPMI medium and incubated (24 hours, 37° C., pH 6.6) with S-labeled ECM (with or without 2.5 μg/ml heparin). The incubation medium was centrifuged and the supernatant analyzed by gel filtration on a Sepharose CL-6B column (0.9×30 cm), as described (9-11, 14). Fractions (0.2 ml) were eluted with PBS and counted for radioactivity using a beta scintillation counter (WALLAC 1409). Degradation fragments of HS side chains were eluted from Sepharose 6B at 0.5<variation of elution positions (Kav)<0.8 (peak II). A nearly intact HSPG was eluted just after the V_{O}, with variation of elution positions (Kav)<0.2, at peak I (9-11, 14). Each experiment was performed at least three times and the Kav values did not exceed +/−15%.

### Experimental Results

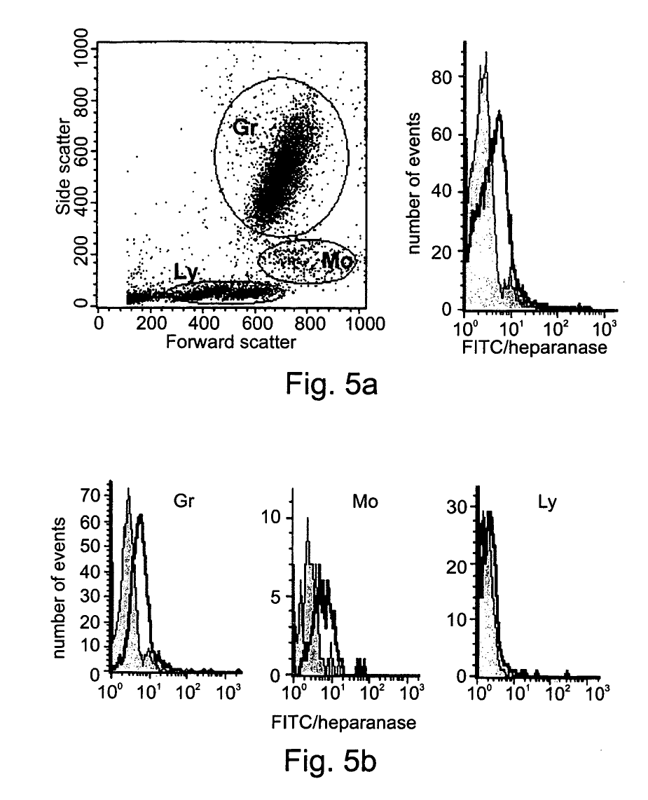

In order to evaluate whether expression of heparanase RNA and protein in AML results in the production of a functional enzyme, representative AML and CLL peripheral blood leukocyte samples were examined for heparanase enzymatic activity. As shown in FIG. 4 (insert), RT-PCR studies confirmed heparanase expression in the AML (lane 1), but not CLL sample (lane 2). Consistent with expression, heparanase activity was confirmed in AML samples as well. As expected, extensive degradation of the heparan sulfates from intact ECM yielded low molecular weight ³⁵S-labeled fragments. This degradative activity was restricted to AML, but not CLL cells (FIG. 4). Degradation of heparan sulfate by AML cells was inhibited in the presence of heparin, indicating specific heparanase activity (FIG. 4) (9-11, 14).

These results indicate that heparanase transcription, translation and activity are characteristic features of AML. Interestingly, some of the CML samples tested, which were also associated with tumor cells of a myeloid lineage, were negative for heparanase mRNA and protein expression.

### Example 4

### High Level Cell Surface Heparanase Expression is Exclusive to Rare Cases of AML

### Material and Experimental Methods

Blood Samples

Peripheral blood and bone marrow samples were obtained from normal and leukemic patients.

Isolation of Peripheral Blood Leukocytes,

Normal and leukemic patient peripheral blood and bone marrow samples were purified by Ficoll-Hypaque (Pharmacia, Uppsala, Sweden) density gradient centrifugation, and then washed twice with PBS as described in example 1, and in (9-11, 14).

Flow Cytometry of Peripheral Blood and Bone Marrow Samples

Samples were stained with the FITC-conjugated anti-heparanase mAb 130, 50 μl samples were incubated with 10 μl mAb 130 (or 10 μl isotype control) for 30 minutes at 4° C. The samples were then incubated with 450 μl erythrocyte lysis solution (Becton Dickinson, San Jose, Calif.) for 12 minutes at 4° C., followed by wash with 2 ml PBS. The samples were then analyzed by flow cytometry utilizing fluorescent activated cell sorting (FACS) (Calibur, Becton Dickinson). Indirect immunofluorescence staining was performed by incubating 0.5-1×10⁶ cells with 10 μg/ml mAb 130 for 30 minutes at 4° C. The samples were washed twice with PBS, followed by incubation with 50 μl of 1:100 diluted PE- or FITC-conjugated goat-anti mouse antibody for 30 minutes at 4° C. The cells were then washed with PBS and analyzed by flow cytometry utilizing FACS Calibur (Becton Dickinson), as well. The total numbers of samples evaluated for heparanase surface expression were: CLL: 10; ALL: 2; AML: 10; CML: 2; myelodysplastic syndrome (MDS): 4; multiple myeloma (MM): 2; lymphoma (LY): 2.

### Experimental Results

Cell-Surface Heparanase Expression is Restricted to Rare Cases of AML

Because the coding sequence of heparanase indicated that the enzyme contained several stretches of hydrophobic amino acids (i.e. Pro515-Ile534) that could potentially serve as membrane anchoring domains (14, 15), it was of interest to determine whether heparanse was expressed on cell surfaces. Initial immunocytochemical staining revealed that heparanase was predominantly a cytoplasmic protein (FIG. 2, 3), though membrane staining might have been obscured by this method of detection. Therefore, FACS analysis was utilized to obtain more definitive results.

Normal peripheral blood samples directly probed with mAb 130, revealed modest cell surface staining for heparanase protein (FIG. 5A). Cell-surface heparanase expression was determined specifically for the three major types of circulating leukocytes, utilizing the characteristics of size (forward scatter) and granularity (side scatter) for sorting the populations: granulocytes (found in region Gr, FIG. 5A), monocytes (found in region Mo, FIG. 5A) and lymphocytes (found in region Ly, FIG. 5A). Sorting for these populations individually, and analysis of fluorescence revealed very low levels of surface heparanase expression detected on granulocytes (FIG. 5B, left) and appreciably less detected on monocytes (FIG. 5B, middle). No significant amount of heparanase was detected on the cell surface of lymphocytes (FIG. 5B, right).

Cell-surface heparanase expression in samples obtained from patients with hematoncological disorders was similarly evaluated (FIG. 6). Cell-surface heparanase expression was detected by FACS in 2 out of 10 AML samples, but not in any of the other 22 samples analyzed, comprising patients with hematological disorders as follows: 2 ALL, 2 CML, 10 CLL, 4 MDS, 2 NHL and 2 multiple myeloma samples.

A representative FACS analysis is shown in FIG. 6. Peripheral blood blasts of an AML M4 patient stained positively for surface expression of both CD14 and heparanase (FIG. 6C), and for CD15 (data not shown). In contrast, the patient's normal lymphocyte population, indicated by CD3⁺ staining, was negative for cell-surface heparanase expression (data not shown). A population of cells expressing high level of cell surface heparanase was detected by flow cytometric analysis of a bone marrow sample from the same patient (FIG. 6D left panel). This sample also contained bone marrow resident CD14+ cells that express very low level of cell surface heparanase. Interestingly, bone marrow samples taken from the patient when in clinical remission revealed the disappearance of the population of cells positive for both heparanase and CD14 expression (FIG. 6D, right panel). These results indicated that high-level surface heparanase expression within the bone marrow detected above reflected leukemic cell populations). Thus, factors controlling cell-surface heparanase expression in leukemic cells were not confined to a specific microenvironment within the circulation, or the bone marrow.

Taken together, these data indicated that heparanase transcription, translation and activity were associated with normal myeloid cells, and were retained following malignant transformation of myeloid cells to AML, though not associated with other leukemic cell populations. Based on these findings, it is therefore possible to determine lineage-related heparanase expression (FIG. 7). Heparanase RNA expression was detected in all samples of acute myelogenous leukemia, except for erythroleukemia. Neither in the erythroblastic cell line (K562) nor in mature erythrocytes was heparanase expression detected, indicating a lack of heparanase expression in the erythroid lineage. Heparanase expression was detected in AML cells of both early and late stages of myeloid differentiation (M2, M3, M4, M5 and M7), as well as in mature circulating granulocytes. These data indicate an early heparanase gene expression checkpoint within the myeloid differentiation pathway. Heparanase expression is sustained throughout the differentiation of the myeloid lineage. Accordingly there may exist a checkpoint for heparanase expression early in the monocyte/myeloid/megakaryocyte lineage. CML onset may take place in cells prior to this checkpoint, thus resulting in heparanase negative CML progenies. Cellular activation events may alternatively turn-on heparanase expression in non-expressing cells (e.g., lymphocytes), or induce the release of the enzyme from heparanase expressing cells (e.g., neutrophils). Most consistently, however, is the finding that AML cells, most commonly at an arrested stage of maturation along the differentiation pathway for granulocytes, elaborate high levels of heparanase, an enzyme critical for granulocyte function. These levels exceed that which is produced by normal granulocytes indicating a unique characteristic in AML leukemic cells readily exploited.

Uniquely, cell surface heparanase expression was also demonstrated to be exclusively limited to rare cases of AML.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications mentioned in this specification are herein incorporated in their entirety by reference into the specification, to the same extent as if each individual publication, patent, patent application was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

1. Kjellen L, Lindahl U (1991) Proteoglycans: structures and interactions. Annu. Rev. Biochem. 60:443
2. Bernfield M, Gotte M, Park P W, Reizes O, Fitzgerald M L, Lincecum J, Zako M (1999) Functions of cell surface heparan sulfate proteoglycans. Annu. Rev. Biochem. 68:729
3. David G (1993) Integral membrane heparan sulfate proteoglycans. Faseb J. 7:1023
4. Wight T N (1989) Cell biology of arterial proteoglycans. Arteriosclerosis 9:1
5. Vaday G G, Franitza S, Schor H, Hecht I, Brill A, Cahalon L, Hershkoviz R, Lider O. (2001) Combinatorial signals by inflammatory cytokines and chemokines mediate leukocyte interactions with extracellular matrix J Leukoc Biol. 69(6): 885-92
6. Liotta L A, Rao C N, Barsky S H (1983) Tumor invasion and the extracellular matrix. Lab. Invest. 49: 636
7. Kleiner D E Jr., Stetler-Stevenson W G (1993) Structural biochemistry and activation of matrix metalloproteases. Curr. Opin. Cell Biol. 5:891
8. Ries C, Loher F, Zang, C, Ismair M G, Petrides P E (1999) Matrix metalloproteinase production by bone marrow mononuclear cells from normal individuals and patients with acute and chronic myeloid leukemia or myelodysplastic syndromes. Clin. Cancer Res. 5:1115
9. Vlodavsky I, Mohsen M, Lider O, Svahn C M, Ekre H P, Vigoda M., Ishai-Michaeli R, Peretz T (1994) Inhibition of tumor metastasis by heparanase inhibiting species of heparin. Invasion Metastasis 14:290
10. Vlodavsky I, Eldor A, Haimovitz-Friedman A, Matzner Y, Ishai-Michaeli R, Lider O, Naparstek Y, Cohen I R, Fuks Z (1992) Expression of heparanase by platelets and circulating cells of the immune system: possible involvement in diapedesis and extravasation. Invasion Metastasis 12:112
11. Vlodavsky I, Fuks Z, Bar-Ner M, Ariav Y, Schirrmacher V (1983) Lymphoma cell-mediated degradation of sulfated proteoglycans in the subendothelial extracellular matrix: relationship to tumor cell metastasis. Cancer Res. 43:2704
12. Nakajima M, Irimura T, Nicolson G L (1988) Heparanases and tumor metastasis. J. Cell Biochem. 36:157
13. Parish C R, Coombe D R, Jakobsen K B, Bennet F A, Underwood P A (1987) Evidence that polysaccharides inhibit tumour metastasis by blocking tumour cell-derived heparanases. Int. J. Cancer 40:511
14. Vlodavsky I, Friedmann Y, Elkin M, Aingorn H, Atzmon R, Ishai-Michaeli R, Bitan M, Pappo 0, Peretz T, Michal I, Spector L, Pecker I (1999) Mammalian heparanase: gene cloning, expression and function in tumor progression and metastasis. Nat. Med. 5:793
15. Hulett M D, Freeman C, Hamdorf B J, Baker R T, Harris M. J, Parish C R (1999) Cloning of mammalian heparanase, an important enzyme in tumor invasion and metastasis. Nat. Med. 5:803
16. Dempsey L A, Plummer T B, Coombs S L, Platt J L (2000) Heparanase expression in invasive trophoblasts and acute vascular damage. Glycobiol. 10:467
17. Fairbanks M B, Mildner A M, Leone J W, Cavey G S, Mathews W R, Drong R, Slighton J L, Bienkowski M J, Smith C W, Bannow C A, Heinrikson R L (1999) Processing of the human heparanase precursor and evidence that the active enzyme is a heterodimer. J. Biol. Chem. 274:587
18. Katz B Z, Levenberg S, Yamada K M, Geiger B (1998) Modulation of cell-cell adherens Junctions by Surface Clustering of the N-Cadherin Cytoplasmic Tail. Exp. Cell Res. 243: 415
19. Friedmann Y, Vlodavsky I, Aingorn H, Aviv A, Peretz T, Pecker I, Pappo O. (2000) Expression of heparanase in normal, dysplastic and neoplastic human colon mucosa and stroma. Am. J. Pathol. 157:1167
20. Liotta L A (1986) Tumor invasion and metastases—role of the extracellular matrix: Rhoads Memorial Award lecture. Cancer Res. 46:1
21. Stetler-Stevenson W G, Aznavoorian S, Liotta L A (1993) Tumor cell interactions with the extracellular matrix during invasion and metastasis. Annu. Rev. Cell Biol. 9:541
22. Welgus H G, Campbell E J, Cury J D, Eisen A Z, Senior R M, Wilhelm S M, Goldberg G I (1990) Neutral metalloproteinases produced by human mononuclear phagocytes. Enzyme profile, regulation, and expression during cellular development. J. Clin. Invest. 86:1496
23. Goetzl E J, Banda M J, Leppert D (1996) Matrix metalloproteinases in immunity. J. Immunol. 156:1
24. Barlett M R, Cowden W B, Parish C R (1995) Differential effects of the anti inflammatory compounds heparin, mannose-6-phosphate, and castanospermine on degradation of the vascular basement membrane by leukocytes, endothelial cells, and platelets. J. Leukoc. Biol. 57:207
25. Dotti G, Garattini E, Borleri G, Masuhara K, Spinelli O, Barbui T, Rambaldi A (1999) Leukocyte alkaline phosphatase identifies terminally differentiated normal neutrophils and its lack in chronic myelogenous leukaemia is not dependent on p210 tyrosine kinase activity. Br. J. Haematol. 105:163
26. Kohno T, Katamine S, Moriuchi R, Miyamoto T, Tomonaga M (1996) Activity of Fgr protein-tyrosine kinase is reduced in neutrophils of patients with myelodysplastic syndromes and chronic myelogenous leukemia. Leuk. Res. 20:221
27. Verfaillie C M, Hurley R, Lundell B I, Zhao C, Bhatia R (1997) Integrin-mediated regulation of hematopoiesis: do BCR/ABL-induced defects in integrin function underlie the abnormal circulation and proliferation of CML progenitors? Acta Haematol. 97:40
28. Enright H, McGlave P B (1995) Chronic myelogenous leukemia. Curr. Opin. Hematol. 2:293
29. Matzner Y, Bar-Ner M, Yahalom J, Ishai-Michaeli R, Fuks Z, Vlodavsky I (1985) Degradation of heparan sulfate in the subendothelial extracellular matrix by a readily released heparanase from human neutrophils. Possible role in invasion through basement membranes. J. Clin. Invest. 76:1306
30. Matzner Y, Vlodavsky I, Bar-Ner M, Ishai-Michaeli R, Tauber A I (1992) Subcellular localization of heparanase in human neutrophils. J. Leukoc. Biol. 51:519
31. Mollinedo F, Nakajima M, Llorens A, Barbosa E, Callejo S, Gajate C, Fabra A (1997) Major co-localization of the extracellular-matrix degradative enzymes heparanase and gelatinase in tertiary granules of human neutrophils. Biochem. J. 327:917
32. Weiss R E, Liu B C, Ahlering T, Dubeau L, Droller M J (1990) Mechanisms of human bladder tumor invasion: role of protease cathepsin Br. J. Urol. 144:798
33. Sloane B F, Moin K, Sameni M, Tait L R, Rozhin J, Ziegler G (1994) Membrane association of cathepsin B can be induced by transfection of human breast epithelial cells with c-Ha-ras oncogene. J. Cell Sci. 107:373
34. DePalma L, Delgado P, Werner M (1996) Diagnostic discrimination and cost effective assay strategy for leukocyte alkaline phosphatase. Clin. Chim. Acta 15:83

1-98. (canceled)

99. A method of distinguishing between heparanase expressing hematopoietic cells and heparanase non-expressing hematopoietic cells in subject in need thereof, the method comprising monitoring heparanase expression in hematopoietic cells obtained from the subject and, based on a presence or an absence of said heparanase expression, distinguishing between said heparanase expressing hematopoietic cells and said heparanase non-expressing hematopoietic cells, wherein monitoring said heparanase expression is by monitoring heparanase RNA levels using an assay selected from the group consisting of RT-PCR, chip hybridization, RNase protection, in-situ hybridization, primer extension, Northern blot and dot blot analysis.

100. The method of claim 99, wherein said heparanase expressing hematopoietic cells or heparanase non-expressing hematopoietic cells are peripheral blood cells.

101. The method of claim 99, wherein said heparanase expressing hematopoietic cells or heparanase non-expressing hematopoietic cells are bone marrow cells.

102. The method of claim 99, wherein said heparanase expressing hematopoietic cells or heparanase non-expressing hematopoietic cells are lymph node cells.

103. The method of claim 99, wherein said heparanase expressing hematopoietic cells comprise activated T or B lymphocytes, platelets, granulocytes, macrophages, monocytes, mast cells, megakaryocytes, promegakaryocytes, megakaryoblasts, promyelocytes, metamyelocytes, myelocytes, myeloblasts, monoblasts and dendritic cells.

104. The method of claim 99, wherein said heparanase non-expressing hematopoietic cells comprise hematopoietic stem cells, erythrocytes, normoblasts, erythroblasts and naive T and B lymphocytes.

105. The method of claim 99, wherein said heparanase expressing hematopoietic cells additionally express the surface markers CD2, CD3, CD4, CD7, CD8, CD10, CD11b, CD13 CD14, CD15, CD16, CD18, CD19, CD20, CD22, CD23, CD25, CD28, CD33, CD34, CD38, CD41, CD42a, CD42b, CD45, CD61, CD71, CD103, CD117, HLA-DR, MPO, FMC7 and TdT.

106. The method of claim 99, used to distinguish between heparanase expressing myeloid cells and heparanase non-expressing B lymphoma cells.

107. The method of claim 99, used to distinguish between heparanase expressing myeloid cells (in acute myeloid leukemia) and heparanase non-expressing myeloid cells (in chronic myeloid leukemia).

108. The method of claim 99, used to distinguish between heparanase expressing myeloid cells and heparanase non-expressing B leukemia cells.

109. The method of claim 99, used to distinguish between heparanase expressing acute myelogenous leukemia cells and heparanase non-expressing chronic lymphocytic leukemia cells.

110. The method of claim 99, used to distinguish between heparanase expressing acute myelogenous leukemia cells and heparanase non-expressing non-Hodgkin's lymphoma cells.

111. The method of claim 99, used to distinguish between heparanase expressing acute myelogenous leukemia cells and heparanase non-expressing chronic myeloid leukemia cells.

112. The method of claim 99, used to distinguish between heparanase expressing acute myelogenous leukemia cells and heparanase non-expressing acute lymphocytic leukemia cells.

113. The method of claim 99, wherein said heparanase expressing hematopoietic cells or heparanase non-expressing hematopoietic cells obtained from said subject are further enriched for leukocytes prior to said monitoring said heparanase expression.


SEQUENCE LISTING (2 sequences)

SEQ ID NO: 1 - DNA, 23 bp, Artificial sequence
  Single strand DNA oligonucleotide
ttcgatcccaagaaggaatcaac

SEQ ID NO: 2 - DNA, 24 bp, Artificial sequence
  Single strand DNA oligonucleotide
gtagtgatgccatgtaactgaatc